(19) United States
(12) Patent Application Publication
(10) Pub. No.: US 2006/0182797 A1
(43) Pub. Date: Aug. 17, 2006

(54) Sutained release formulation for venlafaxine hydrochloride

(76) Inventors: Evangelos Karavas, Agios Dimitrios Attikis (GR); Konstantinos Lioumis, Keratea (GR); Stavros Politis, Athens (GR)

Correspondence Address: AKC PATENTS, 215 GROVE ST., NEWTON, MA, 02466, US

(21) Appl. No.: 10/565,322

(22) PCT Filed: Jul. 23, 2004
(86) PCT No.: PCT/GR04/00039

§ 371 Date: Jan. 20, 2006

(30) Foreign Application Priority Data
Jul. 30, 2003 (EP) 03386019.8

(51) Int. Cl. A61K 9/64

(52) U.S. Cl. 424/456

(57) ABSTRACT

The invention provides a sustained release composition that; 1. Is free of initially increased drug delivery that occurs (in sustained release systems containing the water soluble drug venlafaxine HCl, known as burst phenomenon, by using a functional core partially or totally coated by a functional coating layer or film. 2. Delivers the drug substance within 24 hours and is therefore suitable for once daily administration of the said drug substance. 3. Exhibits linearity between the strength dosage form and the (total mass of the dosage form, by proportional increase of the amounts of the drug substance and the excipients in the formulation. 4. Is possible to be divided in smaller doses, without affecting the release of the drug substance. The invention provides a sustained release capsule formulation containing an appropriate number of functional complex mini tablets comprising of: I. A functional core comprising the active ingredient, especially the water-soluble drug Venlafaxine HCl and appropriate excipients. 2. A functional coating layer or film that reduces the initial surface of the core that is available for the release of the water-soluble drug Venlafaxine HClt phenomenon.

### BACKGROUND OF THE INVENTION

The rapid initial release of the water-soluble drug substances from matrix delivery systems is a well-known phenomenon. Venlafaxine HCL is a drug substance that is very soluble in water (more than 1000 mg are dissolved in 1 ml of water), so the said phenomenon is observed when the release of this drug substance from simple matrix systems is studied.

Another parameter very important for the pharmaceutical industry is the achievement of linearity or proportionality between the strength and the formulation mass. Proportionality between the strength and the formulation mass means that as the amount of the active ingredient increases from a lower to a higher strength, the total mass of the excipients increases at the same rate. This is a point of great interest for the pharmaceutical industry because if this kind of linearity is achieved then the procedures of testing and approving the drug product are much shorter in time and less expensive. It is well known that drug delivery from matrix systems that are tablets is highly affected by the geometrical characteristics of the tablet-matrix. This phenomenon prevents the achievement of linearity between the different strengths of a drug product and the total weight of the said dosage form. As the strength gets bigger and the size of the matrix increases the dissolution rate is delayed, so the dosage forms that refer to different strengths of the same dug product do not exhibit the same dissolution profile. Linearity between the strength and the formulation of a dosage form without the release characteristics being influenced is highly desired in the pharmaceutical industry for manufacturing, pharmacokinetic and economical reasons.

An objective of the present study is to provide a sustained release formulation which is free of the increased release of the drug observed at the initial stages of release that occurs in sustained release systems containing water soluble drugs such as venlafaxine HCl, known as burst phenomenon.

Another objective of this study is to provide a sustained release formulation capable of delivering the drug substance within 24 hours and is therefore suitable for once daily administration of the said drug substance.

Another objective of this study is to provide sustained release formulation that exhibits linearity between the strength of the drug formulation and the total mass of the formulation, by proportional increase of the amounts of the drug substance and the excipients in the formulation.

The dosage form described in the present invention may be divided into smaller doses. This is desired in antidepressant medication treatments, where the therapy is tailored for each individual patient requirement.

Little formulation work has been conducted to date in order to overcome both this release problem concerning water-soluble dug substances such as Venlafaxine HCl and at the same time achieve linearity between strength and formulation as described above. Zero order kinetics are considered an optimal rate for drug delivery from sustained release systems. It is very usual though a rapid release of the drug to be observed during the first hours of release. This rapid initial drug release results to significant deviation from the desired zero order kinetics. This deviation affects the drug plasma concentrations resulting to a higher risk of occurrence of side effects, while effectiveness is deteriorated.

EP700289 describes a type of tablet known as osmotic pump.

EP1253910 also describes an osmotic pump.

EP1178780 describes a multiparticulate controlled release selective serotonin reuptake inhibitor (SSRI) formulation for oral administration, which comprises pellets coated with rate-controlling polymer, which allows controlled release of the SSRI over a period of not less than 12 h.

WO0224160 describes a formulation of Long Acting Antidepressant Microparticles.

EP1028718 and EP0797991 describe encapsulated formulations of spheroid particles as sustained release formulation containing Venlafaxine.

EP1157690 describes a sustained release pharmaceutical composition free of food effect. The composition claimed in this patent is a single double-coated tablet made of compressed granules. Venlafaxine is mentioned as an example of drug substance the absorption of which is known to be influenced by food intake. S. Troy et al., Current Therapeutic Research, VOL.58, NO 8, pp 504-514 performed pharmacokinetic studies in order to assess the effect of food intake on the pharmacokinetic disposition venlafaxine and its active metabolite O-desmethoxyvenlafaxine (ODV). In two studies, Venlafaxine sustained release 75 and 150 mg formulations were administered to healthy subjects in a fasted state or a high fat meal. The studies were conducted with a two period cross over study design. The administration of Venlafaxine sustained release 75 or 150 mg capsules with a fat meal did not affect the rate or extent of Venlafaxine absorption compared with administration to the fasting condition.

In all the above-mentioned patents the osmotic pump type or formulations made from microparticles or spheroids are suggested. However, these types of formulations require both higher cost of production and more sophisticated equipment in relation to more conventional types (e.g. tablets) and at the same time more complicated and thus more time consuming production process. On the other hand, the desired zero order release kinetics is not always achieved.

WO9847491 describes an extended release dosage composition in the form of a tablet matrix, comprising of a drug substance and a combination of a hydrophilic and hydrophobic polymer of well-known groups used for controlled drug delivery formulations. In this patent different rations of the hydrophilic-hydrophobic polymer, as well as channeling agents and surfactants are used in order to modify the wettability of the described matrix, in order to combine it with a drug substance of a given solubility in aqueous systems. This patent is not specifically developed for Venlafaxine HCl and the described dosage form cannot provide linearity between the strength and the formulation of the dosage form, without affecting the release characteristics of the drug substance.

None of the above documents teaches or suggests the present invention.

### SUMMARY OF THE INVENTION

The instant invention provides a process for reducing the initial rapid release of the water-soluble drug substance Venlafaxine HCl from the proposed formulation using one or more functional cores coated with a functional coating layer or film that limits the surface of the core(s) that is available for drug release during the initial stages of the drug delivery.

### DETAILED DESCRIPTION

The present invention consists in a multi tablet capsule delivery system.

Each capsule of the said delivery system contains 1-6 mini tablets, containing the pharmacologically active water-soluble substance substance Venlafaxine HCl.

Each one of the said tablets comprises of a functional core, which is partially or totally coated with an appropriate coating agent, so that the surface of the core that is initially available for drug delivery is limited.

The core comprises of
i. The drug substance, Venlafaxine HCl, in a proportion that varies between 10-40% by weight
ii. 40-80% of a gelling agent. This gelling agent can be chosen among; Hydroxypropylmethylcellulose, hydroxypropylcellulose, hydroxycellulose phthalate, poly(ethyleneoxide), polylactic acid, xanthan gum, alginates, sodium and calcium carboxymethylcellulose, carragheen, carbomer, carbopol (oral use), methylhydroxyethylcellulose, propylhydroxyethylcellulose, polyhema, methylcellulose, alginates and other swellable polymers. The swelling agent used in the formulation should preferably be of high viscosity, as the incorporated drug substance is highly soluble in water and the diffusion rate through the gelling agent should be limited, without beholding the drug substance after the desired time window.
iii. 30-60% of a non-swellable (also characterized as monolithic or plastic) agent or system comprising of one or a mixture of water insoluble, non-swelling polymers such as: ethyl cellulose, cellulose acetate propionate, cellulose acetate, poly(ethyl acrylate, methyl metlacrylate, trimethylammonioethyl methacrylate chloride) 1:2:0.1, commerced as Eudragit RS 100, poly(ethyl acrylate, methyl methacrylate, trimethylammonioethyl methacrylate chloride) 1:2:0.2 copolymer, commercially available as Eudragit RL®, polyvinylpyrrolidone acetate, polyvinyl chloride, polyvinyl acetate, polyethylene, and others. The function of these compounds is to limit the swelling rate of the gelling agent and to reduce the penetration of water through the pores that are formed by the swelling of the gelling agent and the diffusion of the drug substance from the core.
iv. A conjugation agent, a surfactant or a polymer that forms bonds between the gelling agent and the non swellable agent, or between the drug substance and the gelling or the non swellable agent, causing interactions between the constituents of the core that limit its swelling properties. Surfactants that are used as conjugation agents are usually anionic, as sodium lauryl sulphate, sodium docusate, sodium cetostearyl sulphate and triethanolamine lauryl sulphate, in proportions 2-12% by weight. Non ionic compounds, such as polysorbates exhibit weak conduction ability, while cationic surfactants do not have such properties. Polymers used as binding agents between the gelling agent and the drug substance are polyvinylpyrrolidone, polyvinyl alcohol and polyvinylpyrroliudone acetate, in proportions of 10-30% by weight.
v. 1-30% by weight of classical excipients such as:
   a. Lubricants and glidants, as Mg, Ca and Zn Stearate, silicon dioxide, talc and stearic acid, or any other insoluble in water lubricant or glidant.
   b. Binders: the binders adopted for the invention should not be feely soluble in water. For example, polyvinylpyrrolidone acetate is preferred over polyvinylpyrrolidone as it obtains sustained release properties and enhances the relevant behavior of the core.
   c. Diluents: any diluents free of disintegrating properties such as talc, dicalcium phosphate and calcium sulphate dihydrate could be adopted.
      The classical excipients used for the preparation of the core should exhibit low solubility in water and free of disintegrant properties.

The core can be obtained by either a direct compression process, or through a wet granulation and compression process.

In order to optimize the cohesiveness of the core a wet granulation process step is essential. The gelling agent(s), the active ingredient, the non-swelling polymers and the conjugation agent(s) are mixed together, comprising the internal phase to be submitted to the wet granulation step. The solvent used for the wet granulation step could be any suitable solvent for use in the manufacture of oral dosage forms. The solvent or mixture of solvents should be able to dilute or disperse the drug substance, the swellable polymers, the non-swellable polymers and the conduction agent, so that the interactions between the above compounds can be developed. Such solvents are ethanol, acetone, isopropyl alcohol, water and mixtures of the said solvents. Alternatively the non-swelling compound is dissolved into an appropriate co-solvent preparing a 5-40% solution or uniform dispersion that is used for the wet granulation step of the rest of the constituents of the internal phase. The conduction agent may also be diluted or dispersed in the granulation fluid. Any diluents or binders may be added in the internal phase.

After drying, the granule mass is mixed with the excipients comprising the external phase (glidants, lubricants and binders) and the granular/powder mixture is compressed into tablets.

The core is partially or totally coated by a coating layer or a coating film that reduces the initial rapid release of the water-soluble drug substance from the core, via two mechanisms.
i. By reducing the surface of the core that is initially available for the release of the drug substance, during the initial stages of the wetting of the dosage form of the said formulation.
ii. By suppressing the core and in particular the swellable gelling agents. This way the penetration of water through the core that causes the diffusion of the drug substance and its rapid release during the initial steps of the wetting of the core is limited and the “burst” phenomenon is restricted.

The coating layer is applied on (as described in FIG. 1):
i. One surface of the core with thickness that ranges between 3-30% of the diameter of the core, providing a two-layer tablet.
ii. Two surfaces of the core with thickness that ranges between 3-30% of the diameter of the core, providing a three-layer tablet.
iii. One surface and the perimeter of the core with thickness. that ranges between 3-30% of the diameter of the core, forming a “cap” that covers the larger part of the core, leaving only one flat surface for the release of the drug substance.

[image]

The coating layer comprises of a polymer and a water-soluble compound. The polymer can be a swelling agent or a non-swelling agent, similar to the ones used for the core. The water soluble compound can be:
i. A water soluble salt such as sodium chloride, sodium bicarbonate, or any other water soluble salt that can be used as an excipient in a solid oral pharmaceutical formulation.
ii. A water soluble small organic compound like mannitol, lactose, sucrose, sorbitol, citric acid or any other water soluble, low relative molecular mass organic compound that can be used as an excipient in a solid oral pharmaceutical formulation.
iii. A water-soluble polymer like polyvinylpyrrolidone, polyvinyl alcohol, low viscosity hydroxyprolylmethyl cellulose, or any other water-soluble polymer that can be used as an excipient in a solid oral pharmaceutical formulation.

During the initial stages of the wetting of the coating layer the water-soluble compounds dissolve rapidly, creating pores through the drug substance can be diffused and released.

The polymer reduces the diffusion of the drug substance by reducing the surface of the core that is available for the dissolution of the drug substance.

The function of the coating layer is time limited with an optimal duration from 0 up to 2-4 hours of the drug release.

The function of the coating layer is advanced and terminated through two different mechanisms, depending on the kind of polymer that is enabled:
i. In the case of swellable polymers the wetting of the polymer causes the formation of moving boundaries delimiting different physical conditions inside the matrix of the coating layer (dry coating material, swollen polymer, dissolved/undissolved polymer). The polymer swells through a swelling front that is followed by a diffusion front, through which soluble compounds can be diffused through the mass of the polymer and be released and an eroding front through which the polymer dissolves into the surrounding fluids. The termination of the function of the coating layer consisting of swellable polymers coincides with the extension of the diffusion layer up to the surface of the core. After that stage the surface of the core that was covered by the polymer layer can be hydrated and the drug substance can be diffused through the swollen polymer.
ii. In the case of non-swellable polymers the termination of the function of the coating layer is achieved by the breaking of the inelastic coating layer due to the swelling of the core as the core is hydrated through the free surfaces and the pores that are created after the soluble compound of the coating layer is dissolved.

The duration of the function of the coating layer is depended on:
i. The composition of the coating layer and more specifically the polymer-soluble compound ratio. The polymer is usually added at a 1:1 to 9:1 ratio to the water-soluble compound.
ii. The thickness of the coating layer
iii. The kind of the polymer and the kind of the soluble compound
iv. In the case of non-swellable polymers the resistance of the coating layer is also depended on the presence and the percentage of plasticizers. The plasticizers are used in a percentage that ranges between 0-10% and increase the elasticity and consequently the endurance of the coating layer. This way the time period that the coating layer is functional can be controlled through another parameter, the percentage of the plasticizer. Plasticizers used in such formulations are polyethylene glycol, triethyl citrate, glycerol, 1,2 propylene glycol.

The coating layer is applied on the core by a compression process, after mixing the excipients that compose it. As a result the coating layer may contain classical excipients used in direct compression processes, such as glidants, lubricants, diluents and binders. In contrast with the formulation of the cores the coating layer may contain disintegrating agents (such as microcrystalline cellulose, pregelatinized starch, sodium starch glycollate and calcium carboxymethyl cellulose) in proportion between 0-5%, as long as these agents enhance the formation of pores through the polymer mass and do not affect the continuity of the coating layer during the early stages of the drug release.

The cores may also be film coated. Similarly to the coating layer, the coating material is functional for a determined period of time that does not exceed the first 4 hours of the drug release from the core. The film coating usually represents from 1.5 to 18%, by weight of the weight of the mini tablet.

The film coating material contains a polymer at a proportion that ranges between 10-80% of the dry mass of the coating material. The said polymer creates a film that covers the core, reducing the surface of the core that is initially available for the dissolution of the drug substance.

The delivery of the drug substance in the initial stages of the wetting of the coated mini tablets is through pores that are created by the dissolution of water soluble compounds that the coating film contains in a proportion that usually ranges from 20-50% by weight.

The polymers that can be used are:
i. Swellable polymers such as those recited above with respect to the formulation of the core.
ii. Non-swellable such as those recited above with respect to the formulation of the core.
iii. pH-dependent polymers that are insoluble in acidic environment (like the gastric fluids), while they dissolve in slightly acidic (pH 4.5-5.5), neutral or slightly basic pH (6.0-8.0). Such polymers are:
   a) Cellulose acetate phthalate, a polymer that dissolves at pH values over 6.4.
   b) Poly(butyl methacrylate, (2-dimethyl aminoethyl) methacrylate, methyl methacrylate) 1:2:1 copolymer, commercially available as Eudragit E®, that dissolves in pH values lower than 5.
   c) poly(ethyl acrylate, methyl methacrylate) 2:1 copolymer, commercially available as Eudragit 30D®, that dissolves in pH values of about 5.5.
   d) poly(methacrylic acid, methyl methacrylate) 1:1 copolymer, commercially available as Eudragit L®, that dissolves in pH values of about 6.7.
   e) poly(methacrylic acid, methyl methacrylate) 1:2 copolymer, commercially available as Eudragit S®, that dissolves in pH values of about 6.7.

The water-soluble compound may be the same as the ones recited above with respect to the water-soluble compounds of the coating layer.

The coating material may also contain classical excipients such as those recited above with respect to the formulation of the core, as well as plasticizers (such as those recited above with respect to the formulation of the coating layer), colourants (e.g. quinoline yellow, indigotine, sunset yeloow), opacifiers (usually titanium dioxide), adhesive agents (such as low viscosity hydroxypropyl methyl cellulose, hydroxypropyl cellulose and polyvinylpyrrolidone), at a total proportion that ranges between 10-50% by weight of the total weight of the dry coating material.

Ethanol, acetone, water isopropyl alcohol, methylene chloride, chloroform or any other pharmaceutically suitable solvent may be used, as well mixtures of the said solvents, as long as can dissolve or uniformly disperse the constituents of tile coating mixture. The solid content of the coating solution or dispersion typically ranges between 3-40% by weight. The dissolution or dispersion of the solid content of the coating material may be optimised by the use of polyethylene glycol in an amount from 0 to 10% by weight of the coating material.

In the case of the film coating the function of the coating is terminated at an optimal time period as said above. The mechanisms that cause the termination of the function of the film coating are:
i. In the case of coating films consisting of swellable polymers the function of the coating is terminated when the diffusion layer reaches the surface of the core. After that stage the surface of the core that was covered by the polymer layer can be hydrated and the drug substance can be diffused through the swollen polymer, similarly to the process recited above for the coating layers.
ii. In the case of coating films consisting of non-swellable polymers the function of the coating is terminated when the swelling of the core breaks the polymer layer, similarly to the process recited above for the coating layers. The diffusion of the soluble compound creates pores through the core can be hydrated and swell.
iii. In the case of pH-dependent polymer films the function of the coating is terminated through two potential mechanisms: firstly the same mechanism that occurs when the non-swelling polymers are enabled and secondly the change of the pH of the aqueous environment throughout the gastrointestinal track.

### PREFERRED EMBODIMENTS

One preferred embodiment is a capsule containing an appropriate number of mini tablets, in a way that linearity between the strength and the total weight of the dosage form is achieved (1 to 6 mini tablets per capsule).

Each tablet comprises:
i. A functional core comprising Venlafaxine HCl, one or more gelling agents, one or more non-swelling agents, one or more conjugation agents and appropriate quantities of classical excipients
ii. A functional coating comprising an enteric film coating containing water-soluble compound.

### EXAMPLES

The following examples illustrate the invention without limiting it:

### Examples Illustrating the Invasion Macroscopically

Example 1.1: a 0 or 00 size capsule containing 1-6 Venlafaxine 25 mg coated mini-tablets

Example 1.2: a 0 or 00 size capsule containing 1-4 Venlafaxine 37.5 mg coated mini-tablets

Example 1.3: a 0 or 00 size capsule containing 1-3 Venlafaxine 50 mg coated mini-tablets

Example 1.4: a 0 or 00 size capsule containing 1-2 Venlafaxine 75 mg coated mini-tablets

### 2) Examples Illustrating the Core

Example 2.1: the following formulation was prepared:

| | Venlafaxine | Venlafaxine | Venlafaxine | Venlafaxine | % in the |
|---|---|---|---|---|---|
| Ingredient | 25 mg core | 37.5 mg core | 50 mg core | 75 mg core | core |
| Venlafaxine HCl (equivelant to | 28.30 | 42.45 | 56.60 | 84.90 | 26.87 |
| 1:1.132 Venlafaxine base) | | | | | |
| Sodium Lauryl Sulphate | 7.37 | 11.06 | 14.75 | 22.12 | 7.00 |
| Eudragit RS 100 | 7.07 | 10.61 | 14.15 | 21.22 | 6.72 |
| Methocel K100 M | 62.06 | 93.09 | 124.12 | 186.18 | 58.92 |
| Magnesium stearate | 0.53 | 0.79 | 1.05 | 1.58 | 0.50 |
| Total | 105.33 | 158.00 | 210.67 | 316.00 | 100.00 |

Manufacturing process: Venlafaxine HCl, Methocel K 100 M®, and SLS are sieved through a 30 mesh sieve and mixed for an appropriate time period until a uniform mixture is formed. This mixture comprises the internal phase of the formulation. Eudragit RS 100® is dissolved in acetone, preparing a wet granulation fluid. The constituents of the internal phase are wet granulated using the wet granulation fluid. The granular mixture is dried to constant weight in an oven at 40° C. (the total content in solvents is estimated using the Loss on drying method as described in the European Pharmacopoeia 3^{rd} Edition and should be less than 1.5%). The dry granule is mixed with the rest of the excipients in a drum mixer and the resulting mixture is pressed into biconvex tablets (almost spherical in shape) of appropriate mass relatively to the strength and hardness using a Killian® tabletting machine. For the Venlafaxine 25 mg cores 5 mm punches were used, for the 37.5 and 50 mg cores 6 mm punches were used, while for the 75 mg cores 7 mm punches were used. The mini-tablets are placed into 00-sized capsules.

Example 2.2: the following formulation was prepared:

| | Venlafaxine | Venlafaxine | Venlafaxine | Venlafaxine | % in the |
|---|---|---|---|---|---|
| Ingredient | 25 mg core | 37.5 mg core | 50 mg core | 75 mg core | core |
| Venlafaxine HCl (equivelant to | 28.30 | 42.45 | 56.60 | 84.90 | 26.87 |
| 1:1.132 Venlafaxine base) | | | | | |
| Sodium Lauryl Sulphate | 5.00 | 7.50 | 10.00 | 15.00 | 4.75 |
| Eudragit RS 100 | 7.07 | 10.61 | 14.15 | 21.22 | 6.72 |
| Methocel K100 M | 57.36 | 86.04 | 114.72 | 172.08 | 54.46 |
| Kollidon SR | 7.07 | 10.61 | 14.15 | 21.22 | 6.72 |
| Magnesium stearate | 0.53 | 0.79 | 1.05 | 1.58 | 0.50 |
| Total | 105.33 | 158.00 | 210.67 | 316.00 | 100.00 |

Manufacturing process: the same manufacturing process as the one recited above with respect to the core of example 2.1. Kollidon SR® is a commercial name for polyvinulpyrolidon acetate and it was added in the internal phase.

Example 2.3: the following formulation was prepared:

| | Venlafaxine | Venlafaxine | Venlafaxine | Venlafaxine | % in the |
|---|---|---|---|---|---|
| Ingredient | 25 mg core | 37.5 mg core | 50 mg core | 75 mg core | core |
| Venlafaxine HCl (equivelant to | 28.30 | 42.45 | 56.60 | 84.90 | 26.87 |
| 1:1.132 Venlafaxine base) | | | | | |
| Sodium Lauryl Sulphate | 6.32 | 9.48 | 12.64 | 18.96 | 6.00 |
| Eudragit RS 100 | 7.07 | 10.61 | 14.15 | 21.22 | 6.72 |
| Methocel K100 M | 56.04 | 84.06 | 112.08 | 168.12 | 53.20 |
| Kollidon SR | 7.07 | 10.61 | 14.15 | 21.22 | 6.72 |
| Magnesium stearate | 0.53 | 0.79 | 1.05 | 1.58 | 0.50 |
| Total | 105.33 | 158.00 | 210.67 | 316.00 | 100.00 |

Manufacturing process: the same manufacturing process as the one recited above with respect to the core of example 2.1. Kollidon SR® is a commercial name for polyvinulpyrolidon acetate and it was added in the internal phase.

Example 2.4: the following formulation was prepared:

| | Venlafaxine | Venlafaxine | Venlafaxine | Venlafaxine | % in the |
|---|---|---|---|---|---|
| Ingredient | 25 mg core | 37.5 mg core | 50 mg core | 75 mg core | core |
| Venlafaxine HCl (equivelant to | 28.30 | 42.45 | 56.60 | 84.90 | 26.87 |
| 1:1.132 Venlafaxine base) | | | | | |
| Sodium Lauryl Sulphate | 8.43 | 12.64 | 16.85 | 25.28 | 8.00 |
| Eudragit RS 100 | 7.07 | 10.61 | 14.15 | 21.22 | 6.72 |
| Methocel K100 M | 53.93 | 80.90 | 107.87 | 161.80 | 51.20 |
| Kollidon SR | 7.07 | 10.61 | 14.15 | 21.22 | 6.72 |
| Magnesium stearate | 0.53 | 0.79 | 1.05 | 1.58 | 0.50 |
| Total | 105.33 | 158.00 | 210.67 | 316.00 | 100.00 |

Manufacturing process: the same manufacturing process as the one recited above with respect to the core of example 2.1. Kollidon SR® is a commercial name for polyvinulpyrolidon acetate and it was added in the internal phase.

Example 2.5: the following formulation was prepared:

| | Venlafaxine | Venlafaxine | Venlafaxine | Venlafaxine | % in the |
|---|---|---|---|---|---|
| Ingredient | 25 mg core | 37.5 mg core | 50 mg core | 75 mg core | core |
| Venlafaxine HCl (equivelant to | 28.30 | 42.45 | 56.60 | 84.90 | 26.87 |
| 1:1.132 Venlafaxine base) | | | | | |
| Sodium Lauryl Sulphate | 8.43 | 12.64 | 16.85 | 25.28 | 8.00 |
| Eudragit RS 100 | 14.15 | 21.22 | 28.29 | 42.44 | 13.43 |
| Methocel K100 M | 35.57 | 53.36 | 71.15 | 106.72 | 33.77 |
| Kollidon SR | 14.15 | 21.22 | 28.29 | 42.44 | 13.43 |
| Magnesium stearate | 0.53 | 0.79 | 1.05 | 1.58 | 0.50 |
| Talc | 4.21 | 6.32 | 8.43 | 12.64 | 4.00 |
| Total | 105.33 | 158.00 | 210.67 | 316.00 | 100.00 |

Manufacturing process: the same manufacturing process as the one recited above with respect to the core of example 2.1. Kollidon SR® is a commercial name for polyvinylpyrrolidon acetate and it was added in the internal phase.

Example 2.6: the following formulation was prepared:

| | Venlafaxine | Venlafaxine | Venlafaxine | Venlafaxine | % in the |
|---|---|---|---|---|---|
| Ingredient | 25 mg core | 37.5 mg core | 50 mg core | 75 mg core | core |
| Venlafaxine HCl (equivelant to | 28.30 | 42.45 | 56.60 | 84.90 | 26.87 |
| 1:1.132 Venlafaxine base) | | | | | |
| HPC | 5.00 | 7.50 | 10.00 | 15.00 | 4.75 |
| Eudragit RS 100 | 7.07 | 10.61 | 14.15 | 21.22 | 6.72 |
| Methocel K100 M | 57.36 | 86.04 | 114.72 | 172.08 | 54.46 |
| Kollidon SR | 7.07 | 10.61 | 14.15 | 21.22 | 6.72 |
| Magnesium stearate | 0.53 | 0.79 | 1.05 | 1.58 | 0.50 |
| Total | 105.33 | 158.00 | 210.67 | 316.00 | 100.00 |

Manufacturing process: the same manufacturing process as the one recited above with respect to the core of example 2.1. Kollidon SR® is a commercial name for polyvinulpyrrolidon acetate and it was added in the internal phase.

The release profiles of the above formulations were tested using a dissolution apparatus with paddles at 100 rpm using 500 ml of a pH 1.2 solution for the first two hours and 1000 ml of phosphate buffer solution for the rest of the test (total duration 24 h).

The results of the dissolution tests are presented in the following table (table 1) and FIG. 2:

TABLE 1
dissolution tests concerning the cores described
in examples 2.1 to 2.6 (% Dissolved)

| | Core of | Core of | Core of | Core of | Core of | Core of |
|---|---|---|---|---|---|---|
| | example | example | example | example | example | example |
| Time | 2.1 | 2.2 | 2.3 | 2.4 | 2.5 | 2.6 |
| 1 | 17.2 | 25.5 | 19.7 | 19.1 | 19.2 | 27.5 |
| 2 | 32.1 | 36.0 | 28.9 | 26.6 | 27.8 | 37.1 |
| 4 | 41.2 | 44.0 | 35.9 | 33.2 | 33.5 | 46.1 |
| 6 | 49.7 | 52.2 | 43.2 | 40.1 | 39.8 | 54.4 |
| 8 | 63.5 | 66.0 | 53.9 | 51.7 | 48.0 | 67.9 |
| 10 | 72.7 | 75.0 | 63.4 | 60.2 | 55.6 | 77.7 |
| 12 | 79.5 | 81.8 | 71.1 | 68.2 | 64.5 | 85.8 |
| 16 | 84.6 | 86.2 | 74.5 | 71.7 | 66.1 | 87.8 |
| 20 | 89.9 | 91.3 | 80.7 | 76.0 | 71.9 | 93.5 |
| 24 | 95.6 | 100.0 | 83.9 | 80.7 | 76.3 | 102.0 |

[image]

Similar results were noticed when the other strengths were tested for the release of the drug substance, as well as when combinations of the above cores were tested.

The cores containing a conjugation agent exhibited lower initial release of the drug substance Venlafaxine HCl, in a degree the ranges between 3-10%.

There is an optimal ratio between the quantities of the swelling polymer, the non-swelling polymer and the conjugation agent. At this ratio the initial release of Venlafaxine HCl from the formulation is reduced, while the drug substance is quantitatively released from the core at the end of the test (set at 24 hours).

### 3) Examples Illustrating the Coating Layer

The following examples of the coating layer are applied using the core described in example 2.1 as a model core, so that the effect of the coating layer on the formulation can be evaluated. The 75 mg core was enabled as a worst case as it is the core with the biggest surface.

Example 3.1: based on the core described in example 2.1 and the following formulation was prepared for the coating layer:

EXAMPLE 3.1

| Coating Layer Constituents | % |
|---|---|
| Cellulose Acetate Propionate | 99.0 |
| Magnesium Stearate | 1.0 |

The constituents of the coating layer are mixed until a uniform powder mixture is prepared. Then the coating layer is applied by compression on the precompressed core. For the two layer and the three layer tablets the coating layer is applied using the same punches as the ones used for the compression of the core. In the case that the perimeter and one side of the core are coated the punch used for the application of the coating layer is of bigger diameter (usually 1 to 4 mm larger than the diameter of the core). Two levels of the thickness of the coating layer were tested, 1.0 and 2.0 mm, as for the effect of the coating layer on the dissolution profile of complex tablets.

Example 3.2: based on the core described in example 2.1 and the following formulation was prepared for the coating layer:

EXAMPLE 3.2

| Coating Layer Constituents | % |
|---|---|
| Methocel E 50LV | 99 |
| Magnesium Stearate | 1 |

Manufacturing process: the same manufacturing process as the one recited above with respect to the coating layers of example 3.1.

Example 3.3: based on the core described in example 2.1 and the following formulation was prepared for the coating layer:

EXAMPLE 3.3

| Coating Layer Constituents | % |
|---|---|
| POLYOX 900000 | 99 |
| Magnesium Stearate | 1 |

Manufacturing process: the same manufacturing process as the one recited above with respect to the coating layers of example 3.1.

Example 3.4: based on the core described in example 2.1 and the following formulation was prepared for the coating layer:

EXAMPLE 3.4

| | |
|---|---|
| Cellulose Acetate Propionate | 79.0 |
| PVP | 20.0 |
| Magnesium Stearate | 1.0 |

Manufacturing process: the same manufacturing process as the one recited above with respect to the coating layers of example 3.1.

Example 3.5: based on the core described in example 2.1 and the following formulation was prepared for the coating layer:

EXAMPLE 3.5

| | |
|---|---|
| Cellulose Acetate Propionate | 74.0 |
| PVP | 20.0 |
| PEG | 5.0 |
| Magnesium Stearate | 1.0 |

Manufacturing process: the same manufacturing process as the one recited above with respect to the coating layers of example 3.1.

Example 3.6: based on the core described in example 2.1 and the following formulation was prepared for the coating layer:

EXAMPLE 3.6

| | |
|---|---|
| Cellulose Acetate Propionate | 71.5 |
| PVP | 17.5 |
| PEG | 10.0 |
| Magnesium Stearate | 1.0 |

Manufacturing process: the same manufacturing process as the one recited above with respect to the coating layers of example 3.1.

Example 3.7: based on the core described in example 2.1 and the following formulation was prepared for the coating layer:

EXAMPLE 3.7

| | |
|---|---|
| Methocel E 50LV | 79.0 |
| PVP | 20.0 |
| Magnesium Stearate | 1.0 |

Manufacturing process: the same manufacturing process as the one recited above with respect to the coating layers of example 3.1.

Example 3.8: based on the core described in example 2.1 and the following formulation was prepared for the coating layer:

EXAMPLE 3.8

| | |
|---|---|
| Methocel E 50LV | 79.0 |
| Lactose | 20.0 |
| Magnesium Stearate | 1.0 |

Manufacturing process: the same manufacturing process as the one recited above with respect to the coating layers of example 3.1.

The release profiles of the above complex systems were tested using a dissolution apparatus with paddles at 100 rpm using 500 ml of a pH 1.2 solution for the first two hours and 1000ml of phosphate buffer solution for the rest of the test (total duration 24 h).

The results of the dissolution tests performed for the most typical of the above systems are presented in the following tables:

TABLE 2
dissolution profile using the coating layer described in example 3.1

| Coating | Kind of | Thickness of | % Release of Venlafaxne HCl | | | | | |
|---|---|---|---|---|---|---|---|---|
| layer | complex system | coating layer | 1 h | 2 h | 4 h | 10 h | 16 h | 24 h |
| Example | Two layer tablets | 1 mm | 15.0 | 28.7 | 38.1 | 70.9 | 82.3 | 96.1 |
| 3.1 | | 2 mm | 14.2 | 28.0 | 37.6 | 70.1 | 81.4 | 95.4 |
| | Three layer tablets | 1 mm | 10.9 | 26.3 | 33.4 | 68.9 | 80.5 | 93.4 |
| | | 2 mm | 11.1 | 26.7 | 32.8 | 67.7 | 80.2 | 93.1 |
| | Coating of the perimeter | 1 mm | 5.4 | 23.7 | 31.8 | 67.5 | 81.0 | 94.2 |
| | and one side of the core | 2 mm | 5.2 | 22.3 | 30.7 | 66.7 | 80.2 | 93.7 |

TABLE 3
dissolution profile using the coating layer described in example 3.2

| Coating | Kind of | Thickness of | % Release of Venlafaxine HCl | | | | | |
|---|---|---|---|---|---|---|---|---|
| layer | complex system | coating layer | 1 h | 2 h | 4 h | 10 h | 16 h | 24 h |
| Example | Two layer tablets | 1 mm | 14.5 | 26.5 | 35.3 | 70.5 | 83.6 | 97.1 |
| 3.2 | | 2 mm | 13.9 | 26.9 | 34.9 | 70.1 | 71.7 | 98.1 |
| | Three layer tablets | 1 mm | 9.8 | 25.3 | 32.7 | 68.9 | 72.3 | 99.1 |
| | | 2 mm | 9.6 | 24.9 | 30.1 | 67.4 | 72.1 | 99.2 |
| | Coating of the perimeter | 1 mm | 5.5 | 20.4 | 29.4 | 65.3 | 78.9 | 93.7 |
| | and one side of the core | 2 mm | 5.2 | 18.0 | 25.3 | 59.8 | 72.3 | 91.7 |

TABLE 4
dissolution profile using the coating layer described in example 3.4

| Coating | Kind of | Thickness of | % Release of Venlafaxine HCl | | | | | |
|---|---|---|---|---|---|---|---|---|
| layer | complex system | coating layer | 1 h | 2 h | 4 h | 10 h | 16 h | 24 h |
| Example | Two layer tablets | 1 mm | 16.2 | 30.1 | 38.7 | 72.4 | 85.1 | 96.7 |
| 3.4 | | 2 mm | 16.1 | 29.4 | 38.4 | 71.0 | 84.2 | 96.4 |
| | Three layer tablets | 1 mm | 12.1 | 28.3 | 35.6 | 70.3 | 85.6 | 97.8 |
| | | 2 mm | 10.1 | 27.2 | 33.9 | 67.9 | 84.7 | 96.8 |
| | Coating of the perimeter | 1 mm | 4.9 | 21.3 | 28.4 | 64.3 | 79.8 | 94.3 |
| | and one side of the core | 2 mm | 4.6 | 18.9 | 27.6 | 63.2 | 76.6 | 92.5 |

TABLE 5
dissolution profile using the coating layer described in example 3.6

| Coating | Kind of | Thickness of | % Release of Venlafaxine HCl | | | | | |
|---|---|---|---|---|---|---|---|---|
| layer | complex system | coating layer | 1 h | 2 h | 4 h | 10 h | 16 h | 24 h |
| Example | Two layer tablets | 1 mm | 15.9 | 24.6 | 36.2 | 70.9 | 84.2 | 95.8 |
| 3.6 | | 2 mm | 16.1 | 24.3 | 31.2 | 68.4 | 82.1 | 96.7 |
| | Three layer tablets | 1 mm | 12.3 | 22.1 | 34.1 | 70.2 | 82.6 | 96.8 |
| | | 2 mm | 10.1 | 22.3 | 28.9 | 65.2 | 79.8 | 92.3 |
| | Coating of the perimeter | 1 mm | 5.2 | 16.4 | 24.3 | 59.8 | 74.5 | 89.1 |
| | and one side of the core | 2 mm | 4.9 | 16.2 | 23.2 | 57.9 | 73.2 | 88.1 |

TABLE 6
dissolution profile using the coating layer described in example 3.8

| Coating | Kind of | Thickness of | % Release of Venlafaxine HCl | | | | | |
|---|---|---|---|---|---|---|---|---|
| layer | complex system | coating layer | 1 h | 2 h | 4 h | 10 h | 16 h | 24 h |
| Example | Two layer tablets | 1 mm | 16.2 | 29.7 | 39.5 | 79.0 | 93.6 | 98.9 |
| 3.8 | | 2 mm | 15.6 | 30.1 | 39.1 | 78.5 | 80.3 | 99.1 |
| | Three layer tablets | 1 mm | 11.0 | 28.3 | 36.6 | 77.2 | 81.0 | 97.8 |
| | | 2 mm | 10.8 | 27.9 | 33.7 | 75.5 | 80.8 | 96.3 |
| | Coating of the perimeter | 1 mm | 6.2 | 22.8 | 32.9 | 73.1 | 88.4 | 99.7 |
| | and one side of the core | 2 mm | 5.8 | 20.2 | 28.3 | 67.0 | 81.0 | 98.1 |

The above results show that the said complex systems can be applied with great flexibility and cause an extensive reduction of the initial drug release. This reduction ranges from 2 to 13.5%, while the duration of the function of the coating layer can also be controlled and manipulated.

### 4) Examples Illustrating the Coating Film

The following examples of the coating films are applied on the core described in example 2.1 as a model core, so that the effect of the coating film on the formulation can be evaluated. The 75 mg core was enabled as a worst case as it is the core with the biggest surface.

Example 4.1: based on the core described in example 2.1 and the following formulation was prepared for the coating film:

EXAMPLE 4.1

| Coating Film Constituents | % |
|---|---|
| Eudragit RS | 50.0 |
| PEG | 5.0 |
| Talc | 15.0 |
| Lactose | 20.0 |
| Magnesium stearate | 10.0 |

Solvents: Acetone, Acetone:Ethanol 1:1

Manufacturing process: The constituents of the coating film are dispersed in the solvent mixture preparing a homogeneous dispersion of 5-15% solid content. Then the coating film is spray-coated on the cores that were preheated at 70° C. The coating process was completed when the film coating of each core reached a weight of 7-10% of the weight of the core. The film coated cores were dried for 2 hours at 40° C.

Example 4.2: based on the core described in example 2.1 and the following formulation was prepared for the coating film:

EXAMPLE 4.2

| Coating Film Constituents | % |
|---|---|
| Ethyl Cellulose | 30.0 |
| HPMC 50 cp | 20.0 |
| PEG | 5.0 |
| Talc | 15.0 |
| PVP | 20.0 |
| Magnesium stearate | 10.0 |

Solvents: Acetone:Isopropanol 1:1

Manufacturing process: the same manufacturing process as the one recited above with respect to the coating film of example 4.1.

Example 4.3: based on the core described in example 2.1 and the following formulation was prepared for the coating film:

EXAMPLE 4.3

| Coating Film Constituents | % |
|---|---|
| Cellulose Acetate Propionate | 30.0 |
| HPMC 50 cp | 10.0 |
| PEG | 15.0 |
| Talc | 15.0 |
| PVP | 20.0 |
| Magnesium stearate | 10.0 |

Solvents: Acetone:Isopropanol 1:1

Manufacturing process: the same manufacturing process as the one recited above with respect to the coating film of example 4.1.

Example 4.4: based on the core described in example 2.1 and the following formulation was prepared for the coating film:

EXAMPLE 4.4

| Coating Film Constituents | % |
|---|---|
| Cellulose Acetate Phthalate | 30.0 |
| Ethyl Cellulose | 10.0 |
| PEG | 15.0 |
| Talc | 15.0 |
| PVP | 20.0 |
| Magnesium stearate | 10.0 |

Solvents: Acetone:Ethanol 1:1, Acetone, Acetone:H20 97:3

Manufacturing process: Manufacturing process: the same manufacturing process as the one recited above with respect to the coating film of example 4.1.

Example 4.5: based on the core described in example 2.1 and the following formulation was prepared for the coating film:

EXAMPLE 4.5

| Coating Film Constituents | % |
|---|---|
| Kollicoat SR 30 D | 60.0 |
| Propylene Glycol | 12.5 |
| Talc | 7.5 |
| PVP | 10.0 |
| Magnesium stearate | 10.0 |

Water

Manufacturing process: the same manufacturing process as the one recited above with respect to the coating film of example 4.1.

Example 4.6: based on the core described in example 2.1 and the following formulation was prepared for the coating film:

EXAMPLE 4.6

| Coating Film Constituents | % |
|---|---|
| Eudragit L | 12.5 |
| Eudragit S | 37.5 |
| Dibutyl sebacate | 5.0 |
| Talc | 15.0 |
| Lactose | 20.0 |
| Magnesium stearate | 10.0 |

Solvents: Acetone:Isopropanol 1:1

Manufacturing process: the same manufacturing process as the one recited above with respect to the coating film of example 4.1.

Example 4.7: based on the core described in example 2.1 and the following formulation was prepared for the coating film:

EXAMPLE 4.7

| Coating Film Constituents | % |
|---|---|
| Eudragit L | 37.5 |
| Eudragit S | 12.5 |
| Dibutyl sebacate | 5.0 |
| Talc | 15.0 |
| PVP | 20.0 |
| Magnesium stearate | 10.0 |

Solvents: Acetone:Isopropanol 1:1

Manufacturing process: the same manufacturing process as the one recited above with respect to the coating film of example 4.1.

TABLE 7
dissolution profiles of the film coated cores
(75 mg core as described in example 2.1)

| | Weight of the | | | | | | |
|---|---|---|---|---|---|---|---|
| | coating film | | | | | | |
| Kind of | as % of the | % Release of Venlafaxne HCl | | | | | |
| coating film | core weight | 1 h | 2 h | 4 h | 10 h | 16 h | 24 h |
| Example 4.1 | 4% | 9.9 | 26.8 | 33.4 | 71.3 | 83.8 | 95.2 |
| | 8% | 6.5 | 24.1 | 34.7 | 75.6 | 88.9 | 102.1 |
| Example 4.2 | 4% | 13.8 | 26.6 | 34.5 | 69.4 | 71.0 | 97.1 |
| | 8% | 8.0 | 24.3 | 35.0 | 76.1 | 85.6 | 100.1 |
| Example 4.3 | 4% | 12.2 | 29.5 | 33.9 | 71.5 | 83.4 | 95.6 |
| | 8% | 5.6 | 18.5 | 26.4 | 66.0 | 84.5 | 100.4 |
| Example 4.4 | 4% | 10.4 | 20.9 | 28.9 | 64.8 | 79.4 | 94.0 |
| | 8% | 4.6 | 18.9 | 27.6 | 63.2 | 76.6 | 92.5 |
| Example 4.5 | 4% | 12.3 | 27.1 | 35.9 | 73.3 | 87.2 | 103.9 |
| | 8% | 8.9 | 23.7 | 32.6 | 69.3 | 83.1 | 98.8 |
| Example 4.6 | 4% | 12.6 | 29.4 | 37.0 | 73.1 | 89.0 | 101.7 |
| | 8% | 8.7 | 26.8 | 34.5 | 72.0 | 85.1 | 102.3 |
| Example 4.7 | 4% | 15.6 | 28.9 | 37.2 | 69.5 | 82.6 | 97.4 |
| | 8% | 7.6 | 25.1 | 32.8 | 66.9 | 81.2 | 95.9 |

The above results show that the coating films can be applied with great flexibility and cause an extensive reduction of the initial drug release. This reduction ranges from about 3 to about 13%, while the duration of the function of the film coating can also be controlled and manipulated.

1. A pharmaceutical dosage form comprising an extended release formulation of the water-soluble drug substance Venlafaxine HCl, comprising a hard gelatin capsule containing a therapeutically effective number of mini tablets comprising of a functional core and/or a functional coating layer or coating film, so that the initial rapid release of the drug substance from the cores is limited.

2. A pharmaceutical dosage form according to claim 1 wherein the cores of the mini tablets are composed of about 10-40% by weight of Venlafaxine HCl, about 40-80% by weight of a gelling agent, about 30-60% by weight of a non-swelling agent, 2-12% by weight of a conjugation agent and 1-30% by weight of classical excipients with the exception of excipients that exhibit disintegrating properties.

3. A pharmaceutical dosage form according to claim 2 wherein the gelling agent is polymer is selected from the group of Hydroxypropylmethylcellulose, hydroxypropylcellulose, hydroxycellulose phthalate, poly(ethyleneoxide), polylactic acid, xanthan gum, alginates, sodium and calcium carboxymethylcellulose, carragheen, carbomer, carbopol (oral use), methylhydroxyethylcellulose, propylhydroxyethylcellulose, polyhema, methylcellulose and alginates.

4. A pharmaceutical dosage form according to claim 2 wherein the non-swelling polymer is selected from the group comprising from ethyl cellulose, cellulose acetate propionate, cellulose acetate, poly(ethyl acrylate, methyl methacrylate, trimethylammonioethyl methacrylate chloride) 1:2:0.1, commerced as Eudragit RS 100, poly(ethyl acrylate, methyl methacrylate, trimethylammonioetlhyl methacrylate chloride) 1:2:0.2 copolymer, commercially available as Eudragit RL®, polyvinylpyrrolidone acetate, polyvinyl chloride, polyvinyl acetate or polyethylene.

5. A pharmaceutical dosage form according to claim 2 wherein the polymers of the core are conjugated by a pharmaceutically accepted conjugation agent, such as sodium lauryl sulphate, sodium docusate, sodium cetostearyl sulphate and triethanolamine lauryl sulphate, that causes the decrease on the swelling properties of the core.

6. A pharmaceutical dosage form as defined in claim 1 wherein the cores are partially coated by a functional coating layer, covering one or two surfaces of the core, or one surface and the perimeter of the core and the thickness of the coating layer ranging between 3-30% of the diameter of the core.

7. A pharmaceutical dosage form as defined in claim 6, wherein the functional coating layer is comprised of a polymer and a water soluble compound, wherein the said polymer and the said water soluble compound are present in a weight ratio of about 1:1 to 9:1.

8. A pharmaceutical dosage form as defined in claim 6, wherein the polymer is either selected from the group consisting of swellable polymers as the ones recited above in claim 3, or from the group consisting of non-swellable polymers, as the ones recited above in claim 4.

9. A pharmaceutical dosage form as defined in claim 6, wherein the water soluble compound is selected either from the group of water soluble salts, such as sodium chloride, sodium bicarbonate or the group of low relative molecular mass organic solid excipients, such as mannitol, lactose, sucrose, sorbitol or citric acid or from the group of water soluble polymers such as polyvinylpyrrolidone, polyvinyl alcohol or low viscosity hydroxyprolylmethyl cellulose.

10. A pharmaceutical dosage form as defined in claim 1 wherein the cores are film coated by a functional coating film, that represent about 1.5 to 18% by weight of the weight of the core, applied to a sufficient thickness to reduce the initial release of the drug substance from the said formulation.

11. A pharmaceutical dosage form as defined in claim 10, wherein the functional coating film is comprised of a polymer in a proportion of 10-80% of the dry coating material and a water soluble compound, in a proportion of 20-50% of the dry coating material.

12. A pharmaceutical dosage form as defined in claim 11, wherein the polymer is selected either from the group consisting of swellable polymers such as the ones recited in claim 3, or from the group consisting of non-swellable polymers such as the ones recited in claim 4 or from the group of pH-depended polymers that are insoluble in acidic environments while they soften or dissolve in neutral or basic environments, such as cellulose acetate phthalate, Poly(butyl methacrylate, (2-dimethyl aminoethyl) methacrylate, methyl methacrylate) 1:2:1 copolymer, commercially available as Eudragit E®, poly(ethyl acrylate, methyl methacrylate) 2:1 copolymer, commercially available as Eudragit 30D®, poly(methacrylic acid, methyl methacrylate) 1:1 copolymer, commercially available as Eudragit L®, poly(methacrylic acid, methyl methacrylate) 1:2 copolymer, commercially available as Eudragit S®.

13. A pharmaceutical dosage form as defined in claim 11, wherein the water-soluble compound is selected from the groups recited above in claim 9.

14. A pharmaceutical dosage form as defined in claim 1 wherein the coating layer or the coating film further comprises a pharmaceutically accepted plasticizer.

15. A pharmaceutical dosage form as defined in claim 1 wherein the coating layer further comprises classical excipients selected from the group of binders, diluents, glidants, lubricants, adhesive agents, opacifiers and colourants.

16. A pharmaceutical dosage form as defined in claim 1 wherein the coating film further comprises classical excipients selected from the groups of and colourants.

17. A pharmaceutical dosage form as defined in claim 1 wherein the coating film is applied from a solution or dispersion of the said polymer and the said water soluble compound in a pharmaceutically acceptable solvent or mixture of pharmaceutically acceptable solvents where the selected constituents of the coating film can be uniformly dissolved or dispersed.

18. A pharmaceutical dosage form as defined in claim 1 wherein the drug substance the gelling agent, the non-swellable polymer and the conjugation agent are wet granulated using a pharmaceutically acceptable solvent or mixture of solvents.

19. A pharmaceutical dosage form as defined in claim 1 wherein the said capsule comprises one to six of the said mini tablets each one containing 25 to 75 mg of the drug substance.

20. A pharmaceutical dosage form as defined in claim 1 wherein linearity between the total weight of the said mini tablets and the strength of the said dosage form is achieved.

21. A pharmaceutical dosage form as defined in claim 1 wherein the dose may be divided by reducing the number of tablets in each capsule.

22. A pharmaceutical dosage form as defined in claim 1, comprising an extended release formulation for once daily administration, which comprises mini tablets partially or totally coated by a coating layer or coating film that is functional only during the first 2-4 hours of the drug release.

23. A method of preparing a drug delivery system for Venlafaxine which comprises: a) preparing the cores containing Venlafaxine HCl according to claim 5 by a wet granulation, drying and compression process, b) applying a functional coating layer on the cores, according to claim 9, using a direct compression process or applying a functional coating film on the cores using a spraying process, c) encapsulating the prepared mini tablets by using an appropriate encapsulating device.
